(19) United States
(12) Patent Application Publication
(10) Pub. No.: US 2003/0149075 A1
(43) Pub. Date: Aug. 7, 2003

(54) Zoniporide mesylate pharmaceutical compositions and processes for improving solubility of zoniporide

(75) Inventor: Daniel Ray Arenson, East Lyme, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/354,288

(22) Filed: Jan. 30, 2003

Related U.S. Application Data
(60) Provisional application No. 60/353,025, filed on Jan. 30, 2002.

(51) Int. Cl. A61K 31/4709; C 07D 4 3/02

(52) U.S. Cl. 514/314; 546/176

(57) ABSTRACT

Formulations containing a sodium hydrogen exchanger-1 (NHE-1) inhibitor for prevention of, inter alia, perioperative myocardial ischemic injury in mammals and a process for increasing the solubility of said inhibitor with methanesulfonic acid.

### REFERENCE TO RELATED AND COPENDING APPLICATIONS

This application claims the benefit of U.S. Provisional Patent Application No. 60/353,025 filed Jan. 30, 2002, the contents of which are hereby incorporated by reference in their entirety.

### FIELD OF INVENTION

This invention relates to lyophilized formulations containing the mesylate salt of the compound of Formula I, N-(5-cyclopropyl-1-quinolin-5-yl-1H-pyrazole-4-carbonyl)-guanidine, (hereinafter “Zoniporide”). The invention also relates to aqueous formulations of Zoniporide mesylate.

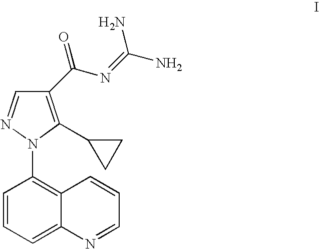

The invention further relates to the process of increasing the aqueous concentration of Zoniporide or Zoniporide mesylate.

### BACKGROUND OF THE INVENTION

Zoniporide is a sodium hydrogen exchanger-1 (NHE-1) inhibitor for prevention of, inter alia, perioperative myocardial ischemic injury in mammals, including humans. Myocardial ischemic injury can occur in outpatient as well as in perioperative settings and can lead to sudden death, myocardial infarction or congestive heart failure. There is an unmet medical need to prevent or minimize myocardial ischemic injury, particularly perioperative myocardial infarction. Such a therapy is anticipated to be life-saving and reduce hospitalizations, enhance quality of life and reduce overall health care costs of high risk patients.

Zoniporide is the subject of co-pending U.S. patent application Ser. No. 09/367,731, filed Aug. 18, 1999, and issued as U.S. Pat. No. 6,492,401, on Dec. 10, 2002; and published as International Application No. WO 99/43663A1 (Attorney docket no. PC10036A). The preparation of Zoniporide and Zoniporide mesylate is the subject of co-pending U.S. patent application Ser. No. 09/636,406, filed Aug. 10, 2000, and issued as U.S. Pat. No. 6,441,176 on Aug. 27, 2002; and published as European Patent Application EP 1101763A2 (Attorney Docket No. PC10656A). The text of the aforementioned published applications and patents, and all other references cited in this specification are hereby incorporated by reference in their entirety.

Lyophilization in commercial vials is a conventional method for preparing pharmaceutical formulations for stability purposes. In the present invention, because of aqueous instability problems, it was determined that Zoniporide could advantageously be stored in the dry state via lyophilization. To achieve the preferred daily dose in a single intravenous bag (about 3 mg/kg/day to about 18 mg/kg/day), however, large vials were necessary, given the inadequate solubility of Zoniporide. It is conventional commercial practice to fill such vials no more than about half full, thereby leaving headspace for shaking once a pharmaceutically acceptable diluent is added to reconstitute the lyophilized formulation as a solution.

Taking into consideration the preferred single dose of Zoniporide, lyophilization techniques and requirements, and cost, the largest commercially feasible vial for lyophilization, therefore, is about 50 mL. Consequently, it was necessary to not only increase the aqueous solubility of Zoniporide, but keep within the constraints imposed by lyophilization, including the limitation of freeze-drying the formulation in 50 mL vials.

In general, formulations of pharmaceutical dosage forms are often hampered by a compound's poor aqueous solubility and stability, which in turn can severely limit its therapeutic application. Methods to increase the solubility and stability of drugs include the use of organic solvents, emulsions, liposomes and micelles, adjustments to pH and the dielectric constant of formulations solvent systems, chemical modifications, complexation of the drugs with appropriate complexing agents, such as cyclodextrins, and formation of different salts of the drug substance.

It was determined that traditional methods of increasing solubility of Zoniporide, or its pharmaceutically acceptable salts, either failed to sufficiently increase solubility or created other problems not acceptable for a pharmaceutical drug. The most commercially feasible and the most pharmaceutically acceptable means, in this particular instance, for increasing solubility of Zoniporide was via pH adjustment. Typical pH adjusters, such as, inter alia, hydrochloric acid, sulfuric acid and phosphoric acid, were considered as a means for increasing the concentration of Zoniporide in aqueous solution, but no significant increase in solubility was measured over a pH range acceptable for infusion.

Surprisingly, however, while conventional pharmaceutical pH adjusting acids were not suitable or optimal, pH adjustment and increase in solubility of Zoniporide could be achieved by the addition of methanesulfonic acid. Further additions of methanesulfonic acid further improved solubility with decreasing pH.

### SUMMARY OF THE INVENTION

In a first aspect, the invention provides a method for increasing the solubility of a compound of Formula I,

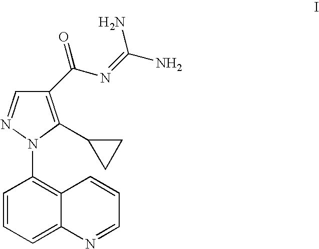

or its mesyalte salt, comprising treating the compound of Formula I with methanesulfonic acid in the presence of an aqueous pharmaceutically acceptable diluent, forming a solution, wherein said solution has a pH in the range of about 2 to about 3.5.

In a preferred embodiment, said pH is adjusted to about 2.2 to about 3.2.

In a preferred embodiment, the method further comprises a molar ratio of methanesulfonic acid to compound of Formula I in the range of 1 to 2.

In a preferred embodiment, the method further comprises a molar ratio of methanesulfonic acid to the mesylate salt of compound of Formula I in the range of 0 to 1.

In a preferred embodiment, said pharmaceutically acceptable diluent is water for injection or 5% dextrose.

In another embodiment, the method further comprises a pharmaceutically acceptable excipient selected from the group consisting of antioxidants, tonicity adjusters, bulking agents, buffers and preservatives.

In a preferred embodiment, said bulking agent is selected from the group consisting of sugars, polyalcohols, amino acids, polymers or polysaccharides.

In a preferred embodiment, said bulking agent is sugar. In a more preferred embodiment, said sugars are selected from the group consisting of glucose, maltose, sucrose and lactose.

In a preferred embodiment, said bulking agent is polyalcohol. In a more preferred embodiment, said polyalcohol is sorbitol or mannitol. In an even more preferred embodiment, said bulking agent is mannitol.

In a preferred embodiment, said bulking agent is an amino acid. In a more preferred embodiment, said amino acid is glycine.

In a preferred embodiment, said bulking agent is a polymer. In a more preferred embodiment, said polymer is polyvinylpyrrolidone.

In a preferred embodiment, said bulking agent is a polysaccharide. In a more preferred embodiment, said polysaccharide is dextran.

In a second aspect, the invention comprises a pharmaceutical composition comprising a mesylate salt of a compound of Formula I,

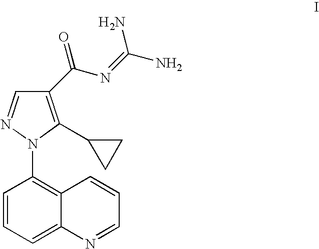

an aqueous pharmaceutically acceptable diluent and methanesulfonic acid, wherein said composition has a pH in the range of about 2 to about 3.5.

In another embodiment, said composition further comprises a pharmaceutically acceptable excipient selected from the group consisting of antioxidants, tonicity adjusters, bulking agents, buffers and preservatives.

In a preferred embodiment, said bulking agent is selected from the group consisting of sugars, polyalcohols, amino acids, polymers or polysaccharides.

In a preferred embodiment, said sugars are selected from the group consisting of glucose, maltose, sucrose and lactose; said polyalcohols are sorbitol or mannitol; said amino acid is glycine; said polymer is polyvinylpyrrolidone; and said polysaccharide is dextran.

In a more preferred embodiment, said bulking agent is mannitol.

In a preferred embodiment, said compositions described above have a pH of about 2.2 to about 3.2.

In a preferred embodiment, said composition has a bulking agent/compound of Formula I ratio in the range of about 1 to about 5.

In a more preferred embodiment, said ratio is in the range of about 1.5 to about 3.

In an even more preferred embodiment, said ratio is in the range of about 1 to about 2.

In a third aspect, the invention provides a pharmaceutical composition comprising a compound of Formula I or the mesylate salt thereof, prepared by lyophilizing said pharmaceutical compositions described above.

In a more preferred embodiment, said lyophilized compositions have a pH in the range of about 2.2 to about 3.2.

In a fourth aspect, the invention provides for a kit comprising:

(a) a therapeutically effective amount of a lyophilized pharmaceutical composition comprising a mesylate salt of a compound of Formula I,

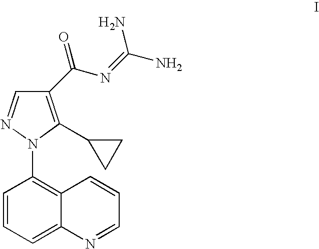

(b) an aqueous pharmaceutically acceptable diluent; and

(c) a first and second container means for containing said composition (a) and said diluent (b), wherein said first container is adapted to receive said diluent from said second container.

In a preferred kit embodiment, said diluent is 5% dextrose.

The term “composition”, as used herein, encompasses, inter alia, (1) solutions comprising the compound of Formula I, Zoniporide, or the mesylate salt of the free base of Zoniporide (hereinafter “Zoniporide mesylate”) or (2) dry lyophilized residues of such solutions. The solutions may contain one or more optional agents which aid in stabilizing dissolved compound of Formula I and/or that facilitate re-dissolution, upon reconstitution of the lyophile created after lyophilizing solution (1). Such optional agents include, inter alia, bulking agents, preservatives, and buffers, as further disclosed herein.

The phrase “increase (or increasing) the solubility” means increasing the solubility of Zoniporide or Zoniporide mesylate to a value greater than the solubility of Zoniporide mesylate in water (i.e. greater than about 18 mgA/mL).

The term “molar ratio” is meant to refer to the moles of methanesulfonic acid to the moles of compound of Formula I, or its mesylate salt.

The unit “mgA/mL” refers to mg/mL of the active compound, Zoniporide.

The term “lyophilization” refers to the conventional, art-recognized procedure freeze-drying a composition. “Lyophilized” and “freeze-dried” are used herein as synonyms.

The term “pharmaceutically acceptable” and the like are meant to refer to applications in both human and veterinary fields.

The term “aqueous pharmaceutically acceptable diluent” is meant to refer to diluents acceptable for intravenous infusion applications in both human and veterinary fields and includes water or other pharmaceutically acceptable excipients for use in making the compositions of the invention (e.g. isotonic solution of sodium chloride, water for injection with ethanol or phosphate, acetate or citrate buffer, and bacteriostatic water for injection with benzyl alcohol).

The term “Zoniporide” means the active compound, Zoniporide, as the free base.

The term “Zoniporide mesylate” means the mesylate salt of Zoniporide made as described in co-pending U.S. patent application Ser. Nos. 09/636,406 (EP1101763 A2), filed Aug. 10, 2000, and 09/657,254 (WO 01/30759A2), filed Apr. 17, 2001. Zoniporide mesylate also means the mono mesylate salt of Zoniporide made by adding methanesulfonic acid to the free base, as described herein, said salt exhibiting a pH of less than or equal to about 5 when dissolved in an aqueous solution.

The term “sugar(s)” is defined herein as including, but not limited to, glucose, maltose, sucrose and lactose.

The term “polyalcohol(s)” is defined herein as including, but not limited to, sorbitol or mannitol.

The term “amino acid” is defined herein as including, but not limited to, glycine.

The term “polymer(s)” is defined herein as including, but not limited to, polyvinylpyrrolidone.

The term ‘polysaccharide(s)” is defined herein as including, but not limited to dextran.

### DETAILED DESCRIPTION OF THE INVENTION

Zoniporide or Zoniporide mesylate can be prepared as known in the art by conventional methodology or as described in co-pending U.S. patent application Ser. Nos. 09/367,731 (WO 99/43663A1), filed Aug. 18, 1999, 09/636,406 (EP1101763 A2), filed Aug. 10, 2000, and 09/657,254 (WO 01/30759A2), filed Apr. 17, 2001, all of which are incorporated herein by reference in their entirety.

With respect to the present invention, stable formulations of Zoniporide mesylate were developed such that a therapeutically effective amount is attained in one or more vials by solubilizing Zoniporide or Zoniporide mesylate with addition of methanesulfonic acid. The invention is further directed toward the process of increasing the solubility of Zoniporide or Zoniporide mesylate by the addition of methanesulfonic acid.

The use of methanesulfonic acid to lower the pH of pharmaceutical formulations is non-traditional in the pharmaceutical field. In particular, methanesulfonic acid surprisingly provided the best concentration of an aqueous solution of Zoniporide, while still maintaining the desired pH range for injectable pharmaceutical formulations. Typically, the pH of pharmaceutical intravenous formulations is not lower than about pH of 3, because of injection-site toleration problems that are inherent in low pH formulations. Surprisingly, however, it was determined that the solubility of Zoniporide could be increased by adjusting the pH of Zoniporide formulations with methanesulfonic acid to as low as about a pH of 2, without the expected injection-site toleration issues. Reducing the pH below about 2, however, can further increase the concentration of Zoniporide, but may cause injection site toleration issues.

For example, in a typical process to solubolize Zoniporide or Zoniporide mesylate for formulations of Zoniporide mesylate, first, methanesulfonic acid is added (e.g. 6 mg/mL), with stirring or agitation, to water (for injection), 5% dextrose or other suitable aqueous pharmaceutically acceptable diluents, containing one or more optional pharmaceutically acceptable excipients, such that the pH is below 5. A therapeutically effective amount of Zoniporide (e.g. 20 mg/mL) or Zoniporide mesylate is then added with agitation until dissolution occurs, (forming Zoniporide mesylate in the case of the addition of Zoniporide). In a preferred embodiment, the pH is adjusted within the range of about 2.0 to about 3.5. Final adjustments to the pH are accomplished by adding methanesulfonic acid or a suitable pharmaceutically acceptable base (e.g. 10% sodium hydroxide).

Aliquots of the resulting formulation solution, the quantity of which is dependent upon the ultimate desired reconstituted concentration of the active compound Zoniporide, are clarified and sterile filtered and aseptically transferred to containers appropriate for lyophilization (e.g. vials), and, in the case of vials, partially stoppered with lyo-stoppers. As described hereinafter, the formulation is cooled to freezing, subjected to lyophilization in a manner conventional per se in the art and hermetically capped, forming a stable, dry lyophile formulation. In a preferred embodiment, said composition has low residual water content, less than 5% by weight, based on the weight of the lyophile. In a more preferred embodiment, said composition has a residual water content level of less than 1% by weight.

Those skilled in the art will appreciate that different orders of addition in making the formulation are possible, other than that described above, without departing from the scope of the invention as defined in the claims. For example, a suspension of Zoniporide or Zoniporide mesylate in a pharmaceutically acceptable diluent containing one or more optional pharmaceutically acceptable excipients may be solubilized by additions of methanesulfonic acid while stirring the suspension, such that the pH is below 5.

In the preferred embodiment, methanesulfonic acid is utilized to adjust the pH to attain sufficient solubility of Zoniporide or Zoniporide mesylate. In general, the amount of methanesulfonic acid in a formulation is such that the molar ratio of methanesulfonic acid to the free base of Zoniporide is in the range of about 1 to 2. The preferred formulation may contain the dissolved mono-mesylate salt and/or dimesylate salt of Zoniporide in varying amounts, dependent upon the amount of methanesulfonic acid utilized and the pH level of the final formulation.

Bulking agents are generally used in lyophilization technology for facilitating the process and/or providing bulk and/or mechanical integrity to the lyophilized cake. As used herein, the word “bulking agent” means a freely water soluble, solid particulate diluent that, when co-lyophilized with Zoniporide mesylate, provides a physically stable lyophilized cake, more optimal freeze-drying process and rapid and complete reconstitution. The bulking agent also may be utilized to make the solution isotonic.

The water-soluble bulking agent suitable for use in the present invention can be any of the pharmaceutically acceptable inert solid materials typically used for lyophilization. Such bulking agents include, for example, sugars such as glucose, maltose, sucrose and lactose; polyalcohols such as sorbitol and mannitol; amino acids such as glycine; polymers such as polyvinylpyrrolidone; and polysaccharides such as dextran. In the preferred embodiment, however, mannitol is used as a bulking agent.

The ratio of the weight of the bulking agent to the weight of active compound, Zoniporide, used in the compositions of the present invention should generally be within the range of from about 1 to about 5. In a preferred embodiment, the ratio of bulking agent to Zoniporide is within the range of about 1 to about 3. The amount of bulking agent is linked to that of Zoniporide. In the preferred embodiment, mannitol is the bulking agent and has a ratio to Zoniporide in the range of about 1 to about 2.

The solubility enhancement effect from the mesylate salt of Zoniporide facilitates the attainment of a solution dosage form having the desired dosage. The lyophilized formulation may contain other excipients known to those skilled in the art such as thickening agents, dispersing agents, buffers, antioxidants, preservatives and tonicity adjusters. Typical buffers include phosphate, acetate, citrate, acetate and glycine. Examples of antioxidants include ascorbic acid, sodium bisulfite, sodium metabisulfite, monothioglycerol, thiourea, butylated hydroxytoluene, butylated hydroxy anisole, and ethylenediaminetetraacetic acid salts. Useful preservatives may include benzoic acid and its salts, sorbic acid and its salts, alkyl esters of parahydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Formulations of Zoniporide mesylate can be manufactured by drying, preferably by lyophilization, as known in the art. Usually, said lyophile formulations are produced with lyophilization (e.g. in a vial) by cooling said formulations at subzero temperature to freezing. The frozen material is then dried under vacuum by subliming the water component originally contained in the solution as a solvent, thus leaving a solid lyophilized cake. Thus, for example, the excipients described above and Zoniporide mesylate are successively dissolved under stirring in a suitable amount of water for injection. The solution is then clarified, sterile filtered and aseptically distributed in sterile containers (e.g. vials) of desired capacity. Freeze-drying is then performed and the vials are hermetically sealed according to conventional procedures.

The isolated dry formulation can be stored at room temperature and reconstituted into a product solution as needed by conventional methods (e.g., with sterile water, water for injection, 5% dextrose, physiological saline solution, or any pharmaceutically acceptable isotonic solution) in an amount sufficient to generate a solution of the required strength for parenteral administration to patients. The injectable reconstituted solutions of the invention are administered either by rapid intravenous injection or preferably by intravenous infusion, according to a variety of possible dose schedules. The formulation may be administered by continuous infusion. The reconstituted compositions of the invention are useful for prevention of perioperative myocardial ischemic injury in mammals, including humans.

Selection of the optimal pharmaceutically acceptable diluent may be dependent upon the concentration of the lyophilized formulation. For example, saline diluents, while appropriate for lower concentrations of reconstituted formulations (e.g. 0.1 mgA/mL) may cause precipitation or incomplete reconstitution of formulations of higher concentrations. For higher concentration formulations, reconstitution with 5% dextrose or water for injection is preferred.

One skilled in the art can appreciate that a variety of dose ranges are possible and are dependent on, inter alia, the subject being treated, the severity of the affliction and the judgment of the prescribing physician. Thus, the below dosages are a guideline and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases (e.g. cardiovascular disease). A preferred dosage is about 0.001 mg/kg/day to 100 mg/kg/day of Zoniporide. An especially preferred dosage is about 0.01 to 50 mg/kg/day, with an even more preferred dosage at about 3 mg/kg/day to about 18 mg/kg/day.

### EXAMPLES

The present invention is illustrated by the following examples, but it is not limited to the details thereof. The formulations express both the final concentration of the active ingredient, Zoniporide, and the concentration of Zoniporide mesylate.

### Example 1

20 mg/mL Zoniporide Formulation

| Components | Concentration (mg/mL) |
|---|---|
| Zoniporide mesylate | 26.00 |
| Mannitol | 40.00 |
| Methanesulfonic acid | 4.56 |

To a compounding vessel, add 90% final weight of water for injection, controlling temperature at 25±50° C. While stirring, add 40 mg/mL mannitol. Stir until dissolved. Add 4.56 mg/mL of methanesulfonic acid. Minimize light exposure and add 26 mg/mL Zoniporide mesylate, stirring until dissolved. Measure the pH and adjust to about 2.2 to about 3.2 pH units with methanesulfonic acid or 10% sodium hydroxide, if necessary. Bring to final volume with water for injection and make final adjustment to a pH of about 2.2 to about 3.2, if necessary, with methanesulfonic acid or 10% sodium hydroxide.

### Example 2

75 mg/mL Zoniporide Formulation

| Components | Concentration (mg/mL) |
|---|---|
| Zoniporide mesylate | 97.5 |
| Mannitol | 120 |
| Methanesulfonic acid | 22.61 |

To a compounding vessel, add 70% final weight of water for injection, controlling temperature at 25±5° C. While stirring, add 120 mg/mL mannitol, stirring until dissolved. Minimize light exposure and add 22.61 mg/mL methanesulfonic acid and Zoniporide mesylate, stirring until dissolved. The pH should be about 1.7. Bring to final volume with water for injection.

### Example 3

100 mg/mL Zoniporide Formulation

| Components | Concentration (mg/mL) |
|---|---|
| Zoniporide mesylate | 130 |
| Mannitol | 150 |
| Methanesulfonic acid | 30.14 |

To a compounding vessel, add 70% final weight of water for injection, controlling temperature at 25±5° C. While stirring, add 150 mg/mL mannitol, stirring until dissolved. Minimize light exposure and add methanesulfonic acid and Zoniporide mesylate, stirring until dissolved. The pH should be about 1.7. Bring to final volume with water for injection.

### Example 4

### Lyophilized Formulation

31 mL aliquots of a solution as prepared above in Examples 1-3 were filled in 50 mL vials and lyophilized using a FTS Kinetics freeze dryer (FTS Systems, Stone Ridge, N.Y.). During lyophilization, the compositions were frozen using a one-step freezing protocol (at −45° C.). The temperature was raised to −10° C. for annealing, then lowered to freezing at −45° C., followed by primary drying at +25° C. for approximately 3400 minutes, followed by a secondary drying with increased steps of temperature to 50° C. The pressure during primary and secondary drying was set at 80 millitorr.

1. A method for increasing the solubility of a compound of Formula I, 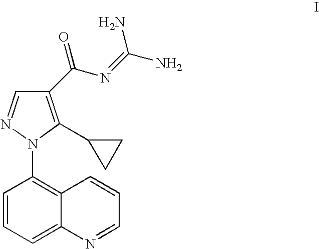
or its mesylate salt, comprising treating the compound of formula I with methanesulfonic acid in the presence of an aqueous pharmaceutically acceptable diluent, forming a solution having a pH in the range of 2 to 3.5.

2. A method according to claim 1, wherein said pH is adjusted to 2.2 to 3.2.

3. A method according to claim 1 or 2, wherein a molar ratio of methanesulfonic acid to the compound of Formula I is in a range of 1 to 2.

4. A method according to claim 1 or 2, wherein a molar ratio of methanesulfonic acid to the mesylate salt of compound of Formula I is in a range of 0 to 1.

5. A method according to claim 1, wherein said pharmaceutically acceptable diluent is water for injection or 5% dextrose.

6. A method according to claim 1, 2 or 5, further comprising a pharmaceutically acceptable excipient selected from the group consisting of antioxidants, tonicity adjusters, bulking agents, buffers, and preservatives.

7. A method according to claim 6, wherein said bulking agents are selected from the group consisting of sugars, polyalcohols, amino acids, polymers or polysaccharides.

8. A method according to claim 7, wherein said sugars are selected from the group consisting of glucose, maltose, sucrose and lactose; said polyalcohols are sorbitol or mannitol; said amino acid is glycine; said polymer is polyvinylpyrrolidone; and said polysaccharide is dextran.

9. A method according to claim 8, wherein said bulking agent is mannitol.

10. A pharmaceutical composition comprising a mesylate salt of compound of Formula I, 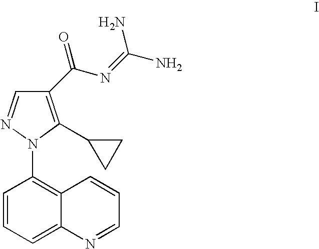
an aqueous pharmaceutically acceptable diluent and methanesulfonic acid, wherein said composition has a pH in the range of 2 to 3.5.

11. A pharmaceutical composition according to claim 10, wherein a molar ratio of methanesulfonic acid to the compound of Formula I is in a range of 1 to 2.

12. A pharmaceutical composition according to claim 10, wherein a molar ratio of methanesulfonic acid to the compound of Formula I is in a range of 0 to 1.

13. A pharmaceutical composition according to claim 10, further comprising a pharmaceutically acceptable excipient selected from the group consisting of antioxidants, tonicity adjusters, bulking agents, buffers and preservatives.

14. A pharmaceutical composition according to claim 13, wherein said bulking agents are selected from the group consisting of sugars, polyalcohols, amino acids, polymers or polysaccharides.

15. A pharmaceutical composition according to claim 14, wherein said sugars are selected from the group consisting of glucose, maltose, sucrose and lactose; said polyalcohols are sorbitol or mannitol; said amino acid is glycine; said polymer is polyvinylpyrrolidone; and said polysaccharide is dextran.

16. A pharmaceutical composition according to claim 15, wherein said bulking agent is mannitol.

17. A pharmaceutical composition according to claims 10, 11, 12, 13, 14, 15 or 16, wherein said pH is 2.2 to 3.2.

18. A pharmaceutical composition according to claims 14, 15 or 16, wherein said composition has a bulking agent/compound of Formula I weight ratio in a range of 1 to 5.

19. A pharmaceutical composition according to claim 18, wherein said ratio is within the range of 1 to 3.

20. A pharmaceutical composition according to claim 19, wherein said ratio is within the range of 1 to 2.

21. A pharmaceutical composition comprising a mesylate salt of compound of Formula I, prepared by lyophilizing said pharmaceutical compositions of claims 10, 11, 12, 13, 14, 15 or 16.

22. A pharmaceutical composition prepared according to claim 21, wherein said composition has a pH in the range of 2.2 to 3.2.

23. A method of reducing tissue damage resulting from ischemia comprising administering to a mammal in need of such treatment a therapeutically effective amount of a composition of claim 21 or a prodrug thereof.

24. A method as recited in claim 23, wherein the tissue is cardiac, brain, liver, kidney, lung, gut, skeletal muscle, spleen, pancreas, nerve, spinal cord, retina tissue, the vasculature, or intestinal tissue.

25. A method as recited in claim 24, wherein the amount of the compound of Formula I is about 0.01 mg/kg/day to about 100 mg/kg/day.

26. A kit comprising:
(a) a therapeutically effective amount of a lyophilized pharmaceutical composition comprising a mesylate salt of a compound of Formula I, 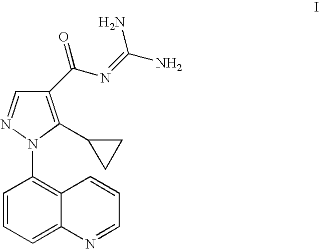
(b) a pharmaceutically acceptable aqueous diluent; and
(c) a first and second container means for containing said composition (a) and said diluent (b), wherein said first container is adapted to receive said diluent from said second container.

27. A kit according to claim 26, wherein said diluent is 5% dextrose.
